(19) United States
(12) Patent Application Publication
(10) Pub. No.: US 2004/0059205 A1
(43) Pub. Date: Mar. 25, 2004

(54) Configuration for monitoring the state of health of a person

(76) Inventors: Sven-Erik Carlson, Herrliberg (CH); Gregor Zund, Herrliberg (CH)

Correspondence Address: NOTARO AND MICHALOS, 100 DUTCH HILL ROAD, SUITE 110, ORANGEBURG, NY, 10962-2100, US

(21) Appl. No.: 10/251,061

(22) Filed: Sep. 20, 2002

(51) Int. Cl. A61B 5/00

(52) U.S. Cl. 600/310; 600/509; 128/904

(57) ABSTRACT

For the acquisition and/or monitoring of medically relevant data, in particular of the state of the cardiovascular system, of blood properties as well as electrocardiographic data of a person, for example with cardiovascular disorders or disorders of other organs, a configuration is proposed, which comprises

at least one measuring sensor (3, 3′) for the acquisition of the circulatory state of the person (1),

at least one electrode configuration (2) for the continuous registration and monitoring of the electrocardiographic data of the person, and

an EKG measuring system (2), respectively electrodes, for the registration of an EKG in a chest belt (4), as well as additionally

an evaluation unit for the detection of irregularities of the data acquired by the measuring sensor or the EKG measuring system,

a sending and receiving device (5) for voice and/or data in order to address a third party (9) and to transmit data to this third party, as well as

a positioning system, by means of which the location of the person is transmitted to the third party.

The present invention relates to a configuration for acquiring and/or monitoring medical data according to the preamble of claim 1, a method for acquiring and monitoring medical data of a person as well as use of the configuration and of the method. The continuous monitoring of the state of health of persons with medical disorders, such as for example cardiovascular disorders or disorders of other organs or other organ functions, is becoming increasingly more important since hospitalization for various reasons is to be avoided.

From DE 197 31 986 the continuous monitoring of electrocardiographic functions of ambulatory patients is known. Similar monitoring configurations are known from U.S. Pat. No. 6,083,248, DE 44 41 907 and WO 01/00085. Therein cardiac function, blood pressure and other data are acquired or registered and transmitted wirelessly to a control center, where potential anomalies or conditions representing health risks are detected and appropriate measures are taken. The proposed monitoring systems permit the monitoring of cardiac function as well as, if appropriate, the diagnosis regarding possible cardiac disease. In all of the described systems no analysis of the oxygen level in the blood in connection with cardiovascular illnesses is carried out. In many cases hypoxemia frequently occurs in patients with acute cardiovascular symptoms and it is therefore reasonable to carry out an oxygen saturation analysis, such as for example by means of pulse oximetry, in order to obtain indications as early as possible of disorders of the cardiovascular system. Hypoxemias of longer duration lead to the diminished supply of cardiac muscle tissue and to cardiac infarction. It is therefore reasonable to diagnose hypoxemias as early as possible in order to lower the mortality.

The not yet published International Patent Application PCT/CH02/00247 proposes in this respect to monitor in particular persons with cardiovascular disorders by means of pulse oximetry, with the measurements being taken by means of pulse oximetry preferably on an ear or on a finger. However, the configuration proposed in this application is only suitable for monitory the state of health of a person and the early detection of health disorders or problems but not for the diagnosis of specific diseases, such as for example the diagnosis of an acute cardiac infarction.

The present invention therefore addresses the problem of proposing a configuration or a method by means of which the continuous monitoring of the state of health or the early detection of a change becomes possible, as well as also a diagnosis in order to be able to draw conclusions regarding potential illnesses, such as for example a cardiac infarction. Early detection of changes of the state of health as well as inquiry of the cardiac condition connected therewith can successfully lower morbidity and mortality as a consequence of cardiovascular illnesses.

The posed problem is solved according to the invention by means of a configuration according to the wording of claim 1. Proposed is a configuration for monitoring which comprises at least the following components:

at least one measuring sensor on the person for the acquisition of medically relevant data, such as in particular data which describe the cardiovascular function and/or contain data regarding blood values or blood composition, which sensor comprises at least one light source which can emit light at least at two frequencies, as well as at least one light receiver for acquiring the light penetrating through a tissue portion or to determine the absorbed or reflected light,

at least one measuring configuration for the continuous registering of an EKG curve,

if necessary, an evaluation unit or digital electronic circuitry for signal processing and interpretation for the sensor or the EKG measuring configuration in order to determine whether or not the measured values are within or outside a defined normal range, or if the EKG curve shows discrepancies from normal curves,

a sending and receiving device for voice and/or data in order to be able to address a third party and to transmit data to this third party as well as, if necessary and optionally

a position determination system which makes possible the precise location such as for example a GPS (Global Positioning System) module, by means of which the location is transmitted to the third party.

The measuring sensors which monitor the state of health of the person, advantageously acquire as many relevant medical data as possible, such as for example the heart rate, respiratory rate, oxygen saturation, cardiac minute volume, blood pressure, blood sugar and if necessary further factors, such as body temperature, etc. The sensors are to be disposed on or in the body such that they ensure maximum freedom of movement and minimum impairment of normal life. All sensors are advantageously disposed in a single sensor unit which can be worn, for example, as a wrist band, as a finger clip, on the ear or subcutaneously. It is understood that this sensor unit can also be disposed on any other body site, on which the above listed medical factors can be measured or determined.

The EKG measuring configuration is provided for the continuous registering of an EKG. It is possible to employ four electrodes according to any known lead or respectively the so-called three-leader, or twelve electrodes according to the preferably to be used twelve-leader. It is known that the so-called twelve-leader yields more precise information about cardiac rhythm as well as about the condition of the cardiac muscle or the EKG registered by the electrodes is more meaningful. For the recording of an electrocardiogram electrodes are normally adhered to the body or fastened with suction cups. In the case of ambulatory situations described according to the invention this is, however, highly impractical and for this reason it is for example proposed to dispose the electrodes in a type of belt, if necessary provided with shoulder straps, which is fixed such that during the wearing the electrodes always remain as much as possible in the same position. Of importance is also that when taking off the belt and putting it back on again, the electrodes are placed again at the same site in order to ensure comparison of the individual EKGs.

Instead of the conventionally utilized electrodes for registering an EKG, more recently light sensors have also been used. The US company Srico Inc., Columbus, Ohio, has recently been offering optic sensor technology for physiological monitoring, especially suitable for registering EEGs and EKGs. Detailed information can be found inter alia at http://www.scrico.com. With the following references to EKG measuring configurations, apart from the conventionally employed electrode configurations, consequently, also optic sensor configurations are understood, wherewith they are also components of the present invention.

The parameters of the physical conditions registered with the sensors or with the EKG measuring configuration are converted into digital signals and supplied to an evaluation system, which checks whether or not the physical condition parameters registered with the sensors or the EKG measuring configuration or the recorded EKG is within or outside of the normal range as defined by a physician of the person or of the patient. If measured values or curve shapes of the EKG outside of the normal range are detected, the evaluation system, by means of a plug connection, wire connection or preferably wireless connection, such as for example a communication in the radio frequency range, outputs a command to a data sending and receiving device for voice and/or data, which the person preferably wears on his body, to establish automatically a connection to the at least one receiver, such as for example to a preprogrammed telephone number or Internet address.

This sending and receiving device can be a mobile telecommunication apparatus, such as for example a so-called GSM telephone (Global System for Mobile communication), a CDMA telephone (Code Division Multiple Access), an UMTS apparatus (Universal Mobile Telecommunication System), AMPS analog system, etc., which apparatus are generally und conventionally utilized as wireless communication means or as replacements for telephone connections via land lines. In principle any mobile telecommunication apparatus can be used, which transmits wirelessly data and/or voice information, be that via a telecommunication network or via the Internet. For the wireless communication between sensor unit and/or EKG measuring configuration as well as the evaluation system and transmission device, such as said GSM telephone, is obvious for application the data communication in the radio frequency range, such as for example the so-called “Bluetooth” technology utilized currently for local voice and data communication, which permits in highly simple manner and utilizing extremely small modules the wireless data exchange between several apparatus. This Bluetooth technology has recently been integrated into said GSM telephone apparatus, which possibly makes superfluous the disposition of a supplementary unit.

The “Bluetooth” technology operates in the 2.4 GHz range and utilizes a relatively complex communication protocol. As a consequence it entails relatively high current consumption. Since current saving is important in the application defined according to the invention, it can be of advantage to utilize a lower frequency and to apply a simpler, specially tailored protocol. Alternatively, the so-called 802.11 (Wireless LAN) protocol can also be used or the Ziq Bee (Home RF Light) protocol which has recently become available, which is a so-called “two-way wireless communication standard” and permits low costs and low current consumption. It is known that the energy consumption at a given voltage is determined by the current amplitude and the duration of the current flow. Consequently energy saving is also possible by transmitting maximally reduced data streams. Without data traffic modern systems can be held in standby with extremely low current consumption. Consequently, it should also be a goal to carry out the evaluation of the signals in as close a proximity to the sensor as is possible, in order to transmit in this case reduced data with short switch-on time if possible relatively noncritical with respect to time, and in the remaining time to set the device to minimum current flow.

In order for a receiver, such as for example a medical specialist or a physician on call in a hospital to know, in addition to the fact that serous health problems occur in the person to be monitored, also where the person is located, the invention proposes to utilize a locating system such as the so-called GPS technology. Mobile telephones for example by the company Benefon (type Esc! and Track), by Motorola (type A820) or Nokia (Type 9200 Communicator) are today offered on the market, which additionally make possible the so-called GPS (Global Positioning System) navigation. Hybrid positioning and transmission as well as information management is moreover ensured by the “Mobile Phone Telematics Protocol” (MPTP). (http://www.wapinsight.com/benefon_gps.htm). Thus, in addition to the EKG itself or data relating to the EKG as well as the data from the sensor system, also position coordinates of the person are transmitted to the receiver, based on which the receiver knows immediately where the person is located. The receiver can either go to the patient himself or for example call up an emergency service provider or an emergency physician in the vicinity of the person.

For more extensive explanations of the properties and advantages of the wireless data or voice signal transmission, reference is made to the above cited, not yet published International Patent Application PCT/CH/00247, which is attached as an annex to this Application.

The EKG measuring configuration listed in addition to the sensor configuration for measuring by means of pulse oximetry, known from said International Patent Application, comprises, as already described above, at least four electrodes, preferably twelve electrodes, in order to make possible the so-called twelve-leader, which supplies more precise information about the condition of the cardiac muscle and therewith makes possible the diagnosis of certain cardiac illnesses. It is proposed that the electrodes are worn by a person in a type of belt, for example provided with shoulder straps, and it is essential that the electrodes are always kept in the same position in order to permit the precise registration of an EKG. The evaluation unit checks continuously whether or not the registered EKG tracing with the P, Q, R, S and T characteristics differ from the personal normal curve. In the event a relevant discrepancy is detected, a corresponding message is wirelessly transmitted to an external office and/or an alarm is triggered at the person. For the interpretation of this alarm, the relative discrepancy is checked against a personal reference EKG as well as potentially also trends are determined in order to be able to detect potential changes of the condition of the patient.

In order to be able to compare meaningfully data from sensor system and EKG measuring configuration to allow the forming of an opinion which goes beyond the possible separate reports from each individual system, it is proposed that the data from both systems can be processed in a central evaluation unit in real time or quasi-real time. The task division of functions between sensor system and EKG measuring configuration, on the one hand, and said evaluation unit, on the other, takes place such that to the evaluation system at least all measured curves/data are available in real time/quasi-real time and at least for the time window required for a reliable assessment of the signal traces, are stored in a local data base.

In order for the state of health to be monitored as much as possible without interruptions for a relatively long time period, it is important that the monitoring system is also ready for use over a long time period and accordingly is supplied with adequate energy. It is consequently a further task of the present invention to propose measures which make possible a period of use of the monitoring system of maximum length or the energy supply of maximum length.

The fact that the signal analysis or evaluation of the data acquired or measured by the sensors is not primarily carried out in the proximity of the sensor(s) or measuring devices, but rather preferably centrally combined in a, for example, separately disposed evaluation unit and/or in or on the sending and receiving unit, as a consequence allows space, weight and battery power to be saved here. This entails the great advantage that the measuring systems or measuring sensors proper can be formed such that they are small and can readily be handled, and that their energy requirement can be reduced to an absolute minium or is extremely low.

According to the invention it is further proposed that the transmission of the data measured by sensor or measuring device to the evaluation unit and/or to the sending and receiving device takes place wirelessly, preferably in the radio frequency range, such as for example by means of the so-called Bluetooth technology.

Said evaluation unit can be worn by the patient himself, such as for example in a belt, a shoulder bag, a backpack, etc. or it can be stationarily disposed separately. The latter is useful especially if the person to be monitored moves within a limited radius of action, such as for example within a house or an apartment. By means of said Bluetooth technology the wireless data transmission is already possible up to a distance of approximately 50 to 100 meters and more recent announced apparatus even permit wireless data transmission in the radio frequency range of up to 200 meters. In this case the evaluation unit consequently can even be connected to a local power line, and simultaneously preferably rechargeable energy storage devices such as batteries can be charged. In addition, in this case the evaluation unit can be connected with a land line sending and receiving unit, such as with a telephone apparatus, an Internet dial-up unit, etc. The transmission of the signals generated by the evaluation unit on, for example, the land line telephone apparatus, can take place either via a connection cable or again wirelessly, for example in said radio frequency range. According to a further embodiment variant, the evaluation unit can be disposed in or on a mobile telephone. That means, for example, that the evaluation unit can be connected such that it can be plugged into a mobile telephone, in order to form a single apparatus, or it can be disposed integrally directly in the mobile telephone.

The combining of the diverse evaluations of the data measured by the sensors or measuring devices in one unit separate from the sensors has the advantage that data from additional monitoring or measuring devices can, of course, be checked or monitored in real time or quasi-real time. It is possible, for example, apart from said measurement by means of pulse oximetry and the registering of an EKG, to monitor also further parameters on a person, such as for example, chest pressure (chest effort), an EEG, in addition to cardiac function the function of additional organs, blood sugar, oxygen level, etc. From these additional measuring sensors the data transmission to the central evaluation unit can also take place wirelessly such as for example in the radio frequency range.

Again a further advantage of such a separate evaluation unit lies therein that based on the registered information about the state of health, automatically completed/controlled auxiliary measures can be taken. For example, upon detecting an insufficient oxygen level in the blood, the O₂ supply via a hose, adapted in terms of quantity, from a portable container can be ensured. Due to the continuous measurement and the comparison of the INSTANTANEOUS/REFERENCE comparison in the evaluation unit, the oxygen supply can be dosed precisely in order to approximate the REFERENCE value as closely as possible with minimum O₂ supply.

In the following such a situation is depicted schematically in the Figures.

Analogously, the blood sugar level can be monitored and regulated by means of, for example, a measuring instrument with the designation “GlucoWatch” by the company Zyanus Inc. or Medtronic Inc. MiniMed Monitor or similar systems, provided, these measuring instruments are equipped with, for example, Bluetooth communication for the purpose of communication with the evaluation unit, such that insulin is automatically injected from a separately disposed injection or dosing device when the blood sugar level in the blood falls below a certain limit.

The signals or data generated by the processing or evaluation unit are subsequently transmitted to a data transmission device, such as a mobile telephone, a stationary telephone apparatus, an Internet dial-up station, etc.

Evaluation of the data acquired by the sensors or measuring devices can take place directly in an apparatus on the person, in an evaluation unit disposed separately in the proximate environment of the person, but also in a help center, into which the data are wirelessly transmitted.

Primarily the curve segments customarily meaningful in cardiology are analyzed such as the QRS complex, the R interval, the S-T segment and the P-Q segment Therein signify:

| | |
|---|---|
| P: | depolarization atria |
| QRS: | depolarization ventricles |
| T: | repolarization atria and ventricles |
| P-Q: | conduction atria to ventricles |
| R-R: | indication of rate and type of rhythm |

The evaluation unit automatically carries out the analysis of the curves in real time, which a cardiologist otherwise completes daily in his work when recording and interpreting EKGs.

It is important that, apart from the constitutional state analysis of a person by means of EKG, a continuous check is also carried out of the extent to which the electrodes are fixed at the correct site. This is made possible thereby that a correct reference state is defined and that it is electronically determined. By checking the voltage it is subsequently possible to check whether or not the electrodes are fixed at the correct site, or if, for example, the halter belt, in which the electrodes are disposed, has changed position. If it is determined that the configuration is not optimally fixed, a message with comments of a change are sent to the mobile telecommunication apparatus. There the message is indicated and sent further to the help center, which, if appropriate, can also convey further instructions to the person wearing the device.

It is important that specifically regarding questions of liability and qualification of the system a journal is kept in the help center listing specific events. This so-called “event log” states for each monitored patient when the EKG monitoring system was activated (thus should have been functioning) and when, due to any influences such as for example activities by the patient, the monitoring by means of the EKG electrode system was not possible.

Specific events which absolutely must be entered, are for example:

the electrode monitoring system or the halter belt is worn and activated—this event is entered into the event log

the belt is not fixed in the correct position: a message is output to the patient, for example via mobile telephone indication; entry into event log

the belt is fixed correctly: again the message is sent to the patient, for example by means of mobile telephone indication—entry into the event log

the electrode configuration or the belt is pulled off: message to patient, for example by means of mobile telephone indication—entry into event log

batteries in belt must be replaced within a certain time period: message to patient for example by means of mobile telephone indication—entry into event log

batteries in mobile telephone must be charged within a certain time period: message to patient, for example by means of mobile telephone indication—entry into event log

there is no radio contact between EKG measuring configuration, evaluation system and mobile telephone: message to patient, for example by means of mobile telephone indication—if appropriate, additional acoustic alarm on halter belt—entry into event log

radio contact established again: message to patient, for example by means of mobile telephone indication—entry into event log

mobile telephone is switched off—entry into event log

monitoring by means of EKG measuring configuration or registration of the EKG is interrupted: entry into event log

etc.

As has already been stated above, the EKG data registered by the EKG measuring configuration or the data transmission from the EKG measuring configuration to the evaluation system and/or, if appropriate, from the evaluation system to the mobile wireless communication transmission unit, preferably takes place in the radio frequency range in analog or digital form, preferably in digital form. In the mobile telecommunication apparatus a so-called evaluation module can be provided, which carries out the analysis of the curves of the EKG in real time or quasi-real time. In this case the connection by means of radio frequency is more or less continuously active.

For considerations of energy it can also be indicated to carry out the signal analysis and interpretation in the proximity of the sensor. If it is completed in an evaluation apparatus associated with a telecommunication apparatus, such as a GSM apparatus, the real-time data stream must be carried via the Bluetooth connection, i.e. the radio module is actively transmitting according to the sampling rate with a high switch-on time component. In contrast, the switch-on time with sensor-proximate evaluation can possibly be significantly reduced if only a reduced data quantity must be transmitted in a manner noncritical as to time.

However, the option is also given that the evaluation unit is also disposed in the belt module. In this way, the connection by means of radio frequency needs only be active when the evaluation unit concludes that in the curve analysis anomalies or discrepancies are present and that an alarm must be triggered in the help center.

In the preferred variant, the evaluation unit is integrated into the mobile telecommunication module or can be plugged onto it and continuously receives in real time or quasi-real time EKG data and possibly data from other sensors, such as for example pulse oximetry data. A cross comparison of these, as well as also the analyzing of the set of data and comparison with stored reference values permits early detection of tendencies which can lead to endangerment of the state of health.

It is important that if an alarm is triggered not only the current alarm message but all information and data relevant to the help center are being transmitted, such as, for example:

the instantaneous registration from the EKG, which has triggered the alarm with specification of that parameter which was outside the tolerance,

the current reference EKG

the currently running EKG curves are transmitted immediately in real time to the help center in order to be processed there either manually or by means of analytical software

corresponding data, registered by other sensor systems such as for example pulse oximetry data

possibly the medical history of the patient and insurance information, which are stored for example on the SIM card of the mobile telephone or on a separate memory card, such as for example a so-called flash card, such as is used for example in the mobile telephone Sony Ericsson P800. In this connection reference is again made to the International Patent Application PCT/CH/02/00247, which, if it is not yet published, is enclosed with the present Application.

Thereby that the identity of the patient is transmitted to the help center through the telephone number, data from a central data base can be retrieved, such as for example the address of the patient, family members who must be informed, family physician, patient's medical history, insurance information, etc.

Analogously to said International Patent Application, in which primarily monitoring the state of health of a person takes place by means of pulse oximetry measurements for example on the ear or on a finger, the additional monitoring by means of EKG measuring configuration or the registering of an EKG is not only suitable or provided for the monitoring of sick persons but it is understood that the registration of an EKG can also be advantageous for healthy persons, where the continuous acquisition of an EKG in the case of persons involved in athletic activities, in monitoring babies, etc., should be mentioned. In the case of sports monitoring, the signals generated by the EKG measuring configuration or the evaluation electronics can be transmitted, instead of to a mobile communication unit, to a wrist band, which, apart from conventional time displays, also can reproduce the medical sports data from the EKG registration, potentially an extract thereof, or they can be conveyed further to an external monitoring center. However, the data can also be stored in a possibly integrated storage medium and be evaluated at a later point in time.

The great advantage of the disposition of a measuring sensor for measuring relevant data regarding the state of health by means of pulse oximetry as well the utilization of an EKG measuring configuration for establishing an EKG lies therein that as an early indicator the pulse oximetry detects very quickly changes relevant to the state of health and that an accurate diagnosis is possible by registering an EKG. Through supplementary data from oxygen measurements in blood and EKG recording, an early and precise diagnosis of cardiovascular illnesses is possible. In other words, medical data can be acquired, which allow drawing conclusions regarding endangerment of the state of health, for example in the presence of insufficient oxygen saturation in the blood, and it is also possible to establish a diagnosis of cardiac illness. Taking measurements as well as making the diagnosis is possible under ambulatory conditions, i.e. on a person who is not hospitalized and is located anywhere in a location not previously defined or announced.

Further preferred embodiment variants in particular of an ear sensor employed for measurements by means of pulse oximetry are found in the International Patent Application PCT/CH/02/00247, which herewith is included as an integral component of the present Patent Application.

The invention will be explained in further detail by example and with reference to the enclosed Figures. Therein depict:

FIG. 1a and 1b in conjunction with a schematic depiction and a block diagram the principle and operational function of the present invention,

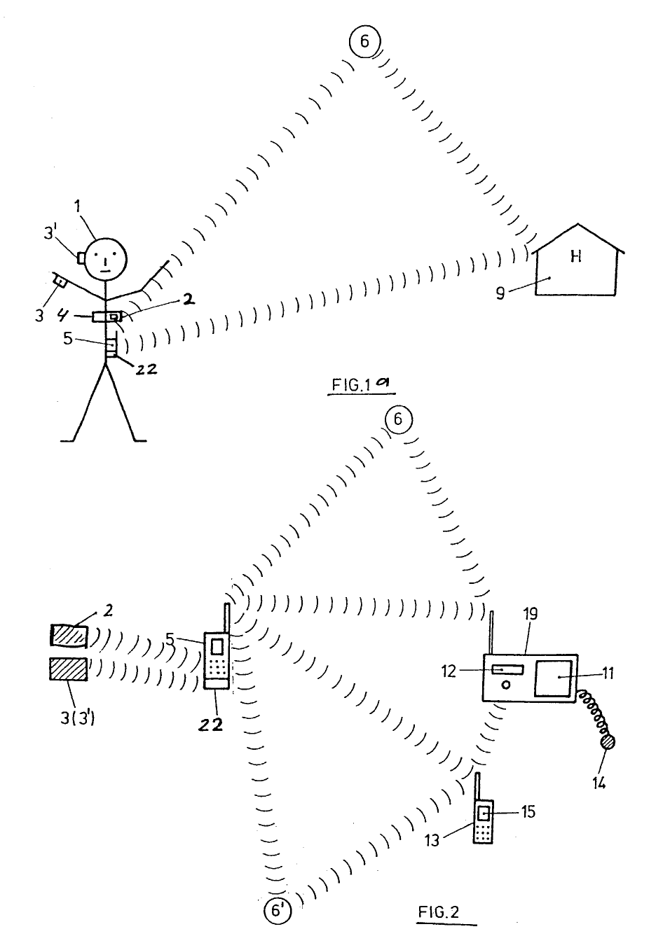

FIG. 2 the individual elements and the functional principle of the present invention in conjunction with a further diagram,

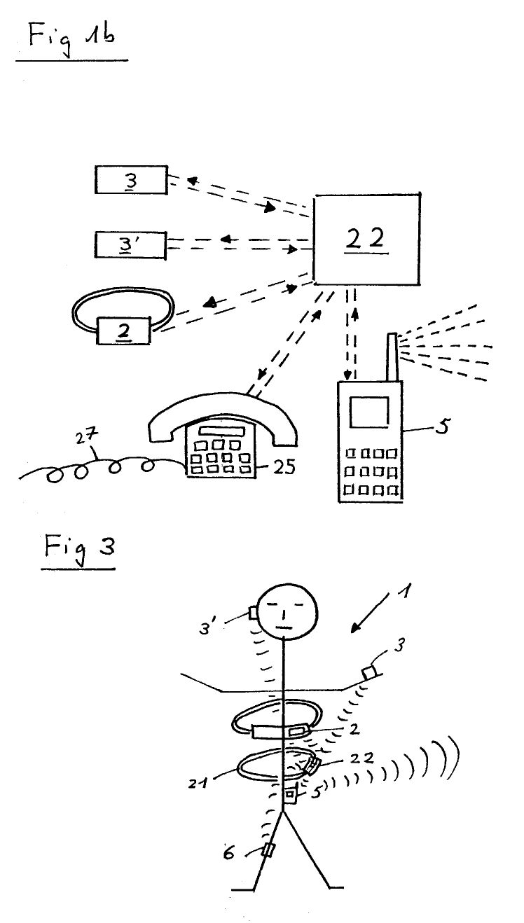

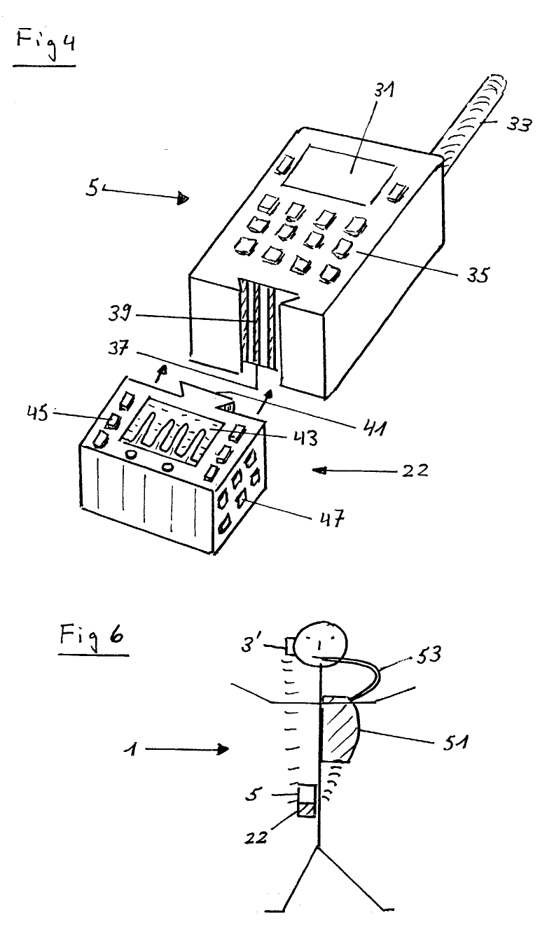

FIG. 3 shown schematically an evaluation and processing unit disposed separately from the sensors or measuring devices for the real-time analysis of the measured data,

FIG. 4 schematically in perspective a preferred embodiment variant of an evaluation and processing unit disposed separately from the sensors or devices, suitable to be plugged onto a mobile telecommunication apparatus,

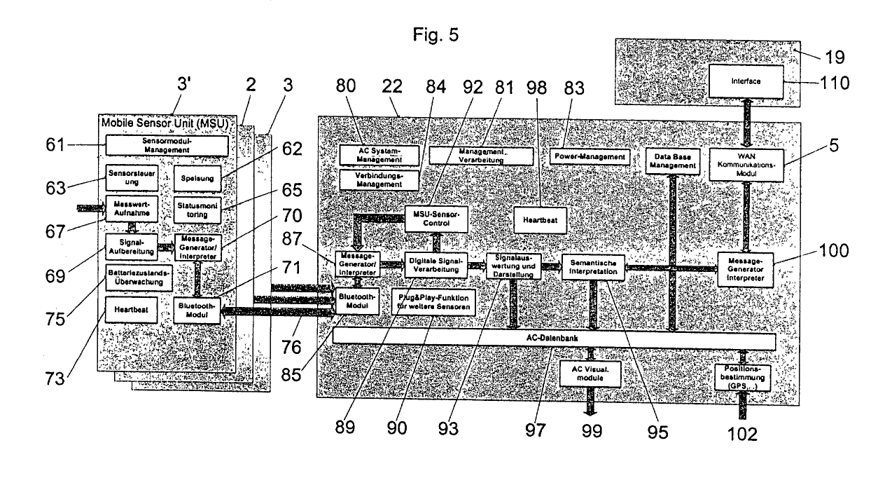

FIG. 5 schematically as a block diagram an interconnection diagram of a feasible embodiment variant of the configuration according to the present invention,

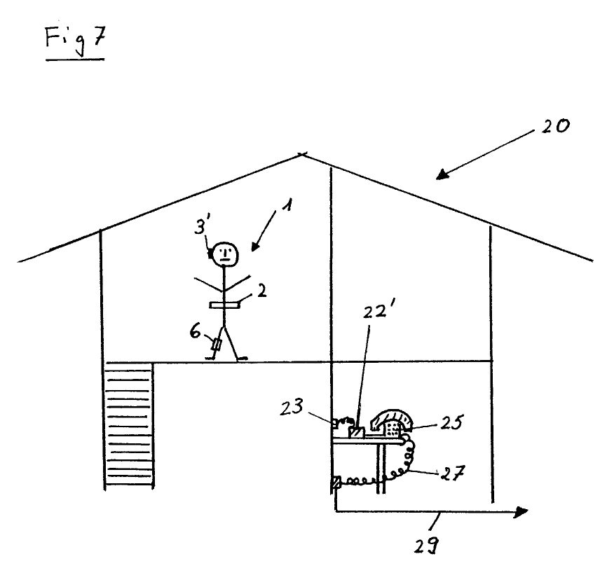

FIG. 6 depicted schematically a monitoring device according to the invention and disposed on a person, who suffers from insufficient oxygen content of the blood, and

FIG. 7 depicted schematically the disposition, distant and separate from the person to be monitored, of the evaluation and processing station, for example when present in a house.

FIGS. 1a and 1b show in conjunction with a schematic representation and a block diagram the principle and the operational function of the present invention. In support of and for better comprehension of the representation of FIG. 1a the different components of the configuration according to the invention are shown schematically in block diagram form in FIG. 1b.

A person 1 is a person with for example cardiovascular disorders. This can be a patient under a physician's care but also a person who has recently been discharged from a hospital, in which he had been hospitalized for example for reasons of a cardiac infarction, or in which he had undergone heart surgery.

But the person 1 can also be a person with risk factors for heart disease.

What is significant is that the person 1 is a person in whom it is suspected cardiovascular problems may shortly occur, which would represent a serious threat to the person 1. For this reason it is important that person 1 stays continuously under medical observation, i.e. that the state of health of person 1 can be continuously monitored.

This is carried out, on the hand, by means of a sensor unit 3 or 3′, which can comprise one or several sensors, by means of which can be monitored for example heart rate, oxygen saturation, blood pressure, cardiac minute volume, body temperature and optionally further factors relevant to health, such as blood sugar level. The sensor unit can be disposed, for example like a wrist band or a finger clip, as shown in FIG. 1 with the reference number 3, or also in conjunction with an ear, as denoted in FIG. 1 by the reference number 3′. In sensor unit 3 are disposed further a radio transceiver, which transmits the registered values continuously to a separately disposed evaluation unit 22 or to a mobile telecommunication unit 5 with integrated or plugged-on evaluation unit 22.

Further is disposed an EKG measuring configuration 2, which is worn by the person 1 for example by means of a chest belt. This EKG measuring configuration is provided for registering electrocardiographic signals such as for example of an EKG. Therein the registration of a so-called three-leader is possible, if four electrodes are emplaced, as well as also the registration of a so-called twelve-leader, if twelve electrodes are emplaced. It is known that the twelve-leader supplies more precise information about the state of the heart and the circulatory system. As described earlier, only the signals measured by the electrode arrangement can be transmitted by means of wireless connection such as for example in the radio frequency range to the evaluation unit 22 or the mobile telecommunication apparatus 5 with integrated or plugged-on evaluation unit 22, but it is also possible to provide in the halter belt 4 an evaluation system connected to the EKG measuring configuration or evaluation electronic circuitry, which continuously monitors the registered EKG and only transmits anomalies with respect to a personal reference EKG to the mobile telephone unit 5 by means of radio frequency.

Continuous transmission of the data to the evaluation unit, which is plugged onto the mobile telecommunication apparatus or is integrated in it, is advantageous since here the analysis of the data from all sensor systems such as for example pulse oximetry and EKG can take place, which yields advantages in the interpretation of disorders.

If irregularities are detected in the analysis, in the mobile telephone, which can be for example a so-called GSM telephone (Global System for Mobile Communication), a dial pulse is triggered by means of which one or several receivers are addressed. The receiver can be for example a rescue center 9 operated for example by a medical specialist. When setting up the connection to the medical specialist via the connection from the mobile telecommunication apparatus 5 to the terminal in a hospital 9, such as a telephone station or an Internet connection, the data are transmitted which had been measured by the measuring unit, such that the specialist on the basis of these data and the identification of the patient, which also is made possible through the mobile telephone 5, can immediately arrive at an assessment of the state of health and, on the basis of the available data, can decide which measures to initiate.

It can be important that the medical specialist knows the position coordinates of the patient 1, in order to know where the patient is located. This can be determined for example by means of the so-called and already widely used GPS system (Global Positioning System), thereby that, in addition to the data transmission, the position coordinates are also transmitted from the mobile telephone 5 via satellite 6 by means of said GPS system. It is understood that other locating configurations are also conceivable, such as for example navigation by means of the GSM network, such as for example the Location Based Service (LSB) which is being offered by the Swiss telecommunication company Swisscom.

The decision must now be made in the rescue center whether a team of the hospital or an external site must be summoned to provide the patient with the necessary help.

It is understood that according to FIG. 1b it is also possible to transmit alternatively to the mobile telephone or simultaneously from the evaluation unit 22 signals or data over a fixedly installed telecommunication device 25, via Internet or via another communication device to an external site, such as for example to a rescue center 9. The transmission can again take place wirelessly or via land line 27.

The present invention or the functional principle will be explained in further detail in conjunction with the diagram of FIG. 2.

As already stated, data acquired by the sensor unit 3 or 3′ or by the EKG measuring configuration are checked in an evaluation or processing system and, in the event of discrepancies from a predetermined measuring range, are transmitted for example wirelessly to a mobile telephone unit 5. The processing or evaluation of the data acquired by the sensors or electrodes can take place in a corresponding real-time or quasi-real time evaluation unit at the sensors or EKG measuring configuration but also in an evaluation unit disposed separately therefrom. Functions of said evaluation and processing unit are inter alia:

Evaluation of measured data, such as for example of registered parameters of physical conditions (if appropriate can also be carried out in the sensor module itself).

Interpretation of the measured data (can also be carried out, if appropriate, in the sensor module).

Establishment of measured value series, administration, storage, correlation, comparative analyzing of the measured data.

To be a point of access for one or several sensor modules.

Generation of messages (reports, data, data series, etc.) to the help center or other sites and transfer of these for example to the mobile communication apparatus, such as a GSM apparatus.

Receiving messages, etc.

For the communication between sensor unit 3 or 3′ or the EKG measuring configuration 2, the evaluation unit 22 and the mobile telephone 5, a wired connection can be established, however, wireless data transmission, such as for example in the radio frequency range, is preferred. The data transmission can also occur by means of infrared or another suitable wireless transmission.

In the event said measured data deviate from the predetermined defined range, the mobile telecommunication apparatus 5 automatically dials a receiver, such as for example a telecommunication apparatus 19, which is connected to a data acquisition and evaluation unit. On it on displays 11 and/or 12 the data or the EKG measured by sensor unit 3 or 3′ and/or of the EKG measuring configuration 2 are reproduced such that a person on duty at the receiver unit 19 can immediately complete an assessment of the health conditions of the patient. By means of the position coordinates of the location of the patient or of the mobile telecommunication apparatus 5, transmitted via a satellite 6 by means of the GPS system, in addition the person on duty can, for example on a screen, immediately determine the location of the patient. Consequently, upon the occurrence of health problems of the patient the medical specialist on duty can virtually without delay immediately initiate the necessary measures to help the patient. It is additionally possible, for example by means of telephone 14, to initiate voice contact with the patient, since by using the mobile telecommunication unit 5 by means of, for example GPRS, Class A, simultaneous voice and data communication is possible. If the patient responds, the medical specialist can for example get information from him about his condition or about his impressions of the situation.

However, it is also possible that the medical specialist automatically receives from the storage medium, which is disposed in or on the mobile telecommunication device, such as the mobile telephone 5, data together with the data measured by the sensor, such as for example the medical history of the patient, or that the medical personnel itself can ask for this information. It is known that each mobile telecommunication device is assigned to a person or a group of persons by an identification chip, such as a so-called SIM card (Subscriber Identity Module). On this module the medical history of the person to be monitored can be stored or, additionally, data important to the medical personnel, such as name and address of the patient, attending physician, family members to be notified, information about medication applications, medical measures already carried out, etc. It is also possible that these data are stored on a memory card plugged in and readily removable, such as for example on a commercially known flash or compact flash card, smart media card, memory stick, etc. This information can additionally decisively affect the necessary measures to be taken.

From the rescue center a further mobile telephone 13 can also be notified, which is carried, for example by the physician treating the patient. On a display 15 of the mobile telephone 13 the measurement data or the EKG, measured by the sensor unit 3 or 3′, or a short version thereof, can also be read off, which can be transferred from the rescue center further to the mobile telephone. The attending physician carrying the mobile telephone 13 can now on his part establish voice contact with the patient. It is understood that the data transmission from the patient takes place directly to the mobile telephone 13 carried by the attending physician, and that the attending physician can also, if necessary, determine the location of the patient thereby that the coordinates are transmitted to him via satellite 6′ by means of GPS. However, as a rule, the contact or the data transmission to a rescue center is obligatory, and the notification to the attending physician takes place depending on the circumstances.

However, with the monitoring system or the configuration proposed according to the invention it is also possible that for example the family physician from time to time via the data communication chain calls up data from the sensor unit 3 or 3′ or from the EKG measuring configuration in order to form an idea about the state of health of a patient.

The monitoring unit proposed according to the invention is also suitable for the self or personal check to acquire sports-related medical data or to be able to call them up. Known are for example measuring instruments worn on a chest belt which are provided to acquire and reproduce heart rate as well as other data, such as running distance, length of time of the sports activity, etc.

FIG. 3 shows schematically a preferred embodiment variant according to the invention of the monitoring configuration, wherein the data registered by the sensors or measuring devices are checked in real time or quasi-real time in a separate processing or evaluation unit apart from the sensors. Analogous to FIGS. 1 and 2, are again provided on a person 1 sensors 3′ or 3, an EKG measuring device 2, as well as a further monitoring device 6, which can be disposed for example on the leg. This additional monitoring device is representative of any further measuring sensors or devices which can be provided for acquiring any medical or other data, which may be significant for the state of health and/or for diagnostic purposes. From these sensors or measuring devices, preferably wirelessly such as for example in the radio frequency range, the data are continuously or periodically conveyed to an evaluation or processing unit 22, which is worn for example in a belt 21 by the person 1. The advantage of this disposition lies therein that this evaluation or processing unit 22 in or on a belt can be larger in terms of dimension and weight than for example in the proximity of the ear sensor 3′. Correspondingly, it is also possible to dispose in this evaluation unit 22 a correspondingly high-energy battery, which can ensure the evaluation of the data transmitted to the unit during a relatively long time period. It is understood that it is also possible to wear this evaluation or processing unit 22 in another form, such as for example in a shoulder bag, in a small backpack, in a trouser pocket provided specifically for this purpose, etc. It is important that an appropriate energy supply of the real-time or quasi-real-time monitoring of the data transmitted to the evaluation unit is ensured and likewise the continuous and/or periodic transmission of the data from the sensors or measuring devices to the evaluation or processing unit 22.

In the event of a discrepancy of the acquired data or in the event irregularities are detected by this evaluation or processing unit 22, a signal is generated or the transmission of corresponding data takes place to the mobile telecommunication 5, from which, analogously to the method described with reference to FIGS. 1a, 1b and 2, the redirection of the data to for example a medical specialist or to a help center takes place.

It is understood that in the sensor unit or EKG measuring configuration a certain signal processing takes place, such that fewer data need to be sent to the evaluation unit. However, under certain conditions, this can be of advantage for saving current.

The evaluation unit, which processes information from the sensor units and the EKG measuring configuration as well as detects alarm conditions, can, as described with reference to FIG. 3, be developed to be separate or as a so-called “stand alone” unit, or be disposed or mounted for example pluggable on a sensor unit or on the EKG measuring configuration. However, it is also possible to dispose the evaluation unit such that it can be plugged onto or in a telecommunication apparatus or to dispose it integrally in the telecommunication apparatus.

In FIG. 4 is shown schematically and in perspective a feasible embodiment variant of a separately developed evaluation or processing unit 22, which can be disposed for example and preferably such that it can be plugged onto a mobile telecommunication unit 5. The mobile telecommunication unit 5 can be a commercially available mobile telephone, with indication display 31, antenna 33 as well as operating keypad 35, on which mobile telephone is additionally developed, for example on a side face, a plug-in section 37, formed in the shape of for example a dove tail. It is understood that this section can also be a plug-in section on which an auxiliary apparatus can be plugged in by means of pins. This part 37 is additionally developed with contact surfaces such as for example strip conductors 39.

The separately disposed evaluation or processing unit 22 can be developed such that it can be plugged in or on the dove-tail like side face 37 of the mobile telephone 5. The corresponding counterpiece on the evaluation unit 22 is schematically provided with the reference number 41. Correspondingly, on this contact faces 41, which can be plugged in or plugged on, again strip conductors are provided which, after the evaluation unit 22 is plugged onto the mobile telephone 5, come into contact with corresponding strip conductors 39 of the mobile telephone 5.

By developing, on the one hand, a mobile telephone as depicted in FIG. 4 and, on the other hand, of evaluation or processing units 22, again as depicted in FIG. 4, it is possible to develop the two apparatus classes differently, to use or interchange them from different manufacturers or to adapt them to specific requirements, etc.

If plugged on, as shown in FIG. 4, it is possible to employ the display from the telecommunication apparatus, such as the mobile telephone, as an information display of the evaluation unit.

As stated earlier, it is understood that it is also possible to dispose the evaluation unit integrally in the telecommunication apparatus. It would furthermore be possible that, if already available in the telecommunication apparatus, such as the mobile telephone, the Bluetooth chip integrated therein could also be used for the data transmission from and to the sensor units and the EKG measuring configuration.

In FIG. 5 is shown schematically in block diagram representation a feasible embodiment variant of a configuration according to the invention. FIG. 5 depicts schematically three sensor units 3′, 2 and 3, which can be for example an ear sensor, the EKG measuring configuration as well as a further sensor. In conjunction with for example the ear sensor 3′, the different components are depicted schematically and in simplified form. The sensor is generally controlled and monitored by the sensor module management unit 61. A feed 62 supplies the necessary energy, which can be for example a battery. The sensor control 63 determines how and when measurements are to be carried out. The status monitoring 65 checks whether or not the measurements are carried out correctly according to the control.

From a measurement value pick-up 67 the measured values are transferred further to a signal processing 69 where the luminous flux measured by the ear sensor is amplified and digitized. From there the measured values arrive at a message generator/interpreter 70, in which the data, for example Bluetooth compatible, are processed so that they can subsequently be transmitted wirelessly from the Bluetooth module 71. Further available in the mobile sensor unit 3′ are a so-called heart beat module, by means of which system monitoring takes place, as well as also by means of the battery status monitoring module 75 the battery status is checked and, if necessary, indicated.

The heart beat module provides two functions:

a) The so-called Watch Dog Function: it monitors the correct functioning of the measuring sensor or the measuring module. The goal is to detect function disturbances due to hardware as well as also software, thereby that a timer, realized in the module periodically through the software, such as for example the control, is reset. If the execution of the software code is blocked, the timer is not reset; it overruns as a consequence and triggers a reset of the measuring sensor. This function operates on every measuring sensor independently of every other measuring sensor.

b) Heart Beat Function: it monitors the communication connection from measuring sensor and evaluation unit in that periodically a connection is briefly set up in order to test the connection. This protective function makes possible that when losing the connection a response can occur with is appropriate to the situation.

Subsequently, the data sent by the Bluetooth module are sent wirelessly, schematically depicted by the double arrow 76, to the corresponding Bluetooth module 85 in the evaluation unit 22. Correspondingly, data are, of course, also transmitted wirelessly from the further configurations or sensors 2 and 3 to the evaluation unit 22. From the Bluetooth module 85 the data are first moved to the message generator/interpreter 87, in which the data received by means of Bluetooth transmission are again transformed back analogously to the message generator/interpreter 70 of mobile sensor unit 3′, in order for them to be utilized for further processing or evaluation. From the message generator/interpreter the data are subsequently transmitted to the digital signal processing 89, where the data are worked up in order to be able to be represented subsequently in the signal evaluation and representation 93 appropriately compatible with the system. Compatible with the system means for example that they are compatible for example with the data of the data base 97 or can be compared with them. The evaluation proper of whether or not a measured value is within a predetermined range or whether or not a disorder is present, takes place in the semantic interpretation unit 95. Apart from the analysis of whether the data are within or outside a predetermined range, a trend analysis can also be completed in the semantic interpretation 95. The visualization proper of the evaluated data or the interpretation takes place by means of the (AC) visualizing module 99.

If the acquired measured data show or if it is revealed in the evaluation that an abnormal state is present, a signal is conducted further to the message generator/interpreter 100, which transmits via the WAN communication module 5 or via, for example, the mobile telecommunication unit the signal to an external monitoring system, at which the signal is received for example via an interface 110. In order for the position of the monitored patient to be detected in the external monitoring unit 19, a position determination module 102 is provided either with the telecommunication module 5 or in the evaluation unit 22.

If by the digital signal processing 89 for example irregularities are detected in the measured values, a feedback via the MSU sensor control unit 92 can take place via which signals are transmitted back to the mobile measuring sensor 3′ in order to affect or adjust for example the measured rhythm, the light intensity, measuring intervals, etc. This can be the case for example if moisture is present on the ear, for example during rain, if the ear temperature is low, such as for example in winter, etc., such that a different light intensity needs to be used for the measurements.

The evaluation unit 22 can further comprise operating modules, such as for example an AC system management 80, a management processing unit 81, a power management 83, a connection management 84, etc. All of these units or modules are provided in order to monitor or regulate the operation, energy supply, system compatibility, etc. of the evaluation unit. For this purpose also serves the heart beat module 98, already known from and provided in mobile sensor unit 3′, which module can be provided for system monitoring.

Lastly, in the evaluation unit 22 a module for plug-and-play for new sensors can be disposed, which automatically acquires new mobile sensor units which transmit data wirelessly to the Bluetooth module 85.

It is understood that the schematic representation in FIG. 5 is only an example which is intended for the purpose of explaining or describing in further detail a feasible embodiment variant of the present invention.

FIG. 6 depicts schematically a concrete situation of the manner in which the configuration proposed according to the invention can be applied meaningfully on a person to be monitored. The person 1 as shown in FIG. 6 is experiencing an insufficient oxygen level in the blood, which can be continuously monitored or detected for example via the ear sensor 3′ by means of pulse oximetry. If the insufficient oxygen level is measured by the sensor 3′ and if the evaluation in the evaluation or processing unit 22 shows an insufficient oxygen level, a quantity-adapted O₂ supply is carried out immediately via a hose 53 from a portable container 51 carried along by the person. The container 51 to be carried along can be carried by the person 1 for example in a backpack. The configuration: ear sensor 3′, evaluation unit 22-oxygen container 51 as well as O₂ supply 53 consequently form a regulation circuit, which is activated when an insufficient oxygen level is detected by sensor 3′.

In other words, in the evaluation unit 22 a regulating circuit logic can additionally be provided, which already checks or regulates on the person to be monitored possible measures, such as said oxygen supply.

By means of the mobile telecommunication unit 5 a signal or information can additionally be conducted to an external site as a warning signal when the oxygen level falls below a limit or if, on the one hand, the oxygen level becomes critical in spite of regulation, or if, for example, no more oxygen is available in the container, etc.

Analogously, the blood sugar level can also be detected and insulin can automatically be injected if the level falls below a certain limit. If the person to be monitored possesses for example a heart pacemaker, it can even be possible that when irregularities are detected by the EKG measuring configuration certain function parameters of the heart pacemaker are affected

A great advantage of this independent additional evaluation unit 22 lies therein that it can also be disposed remote from the person 1 and yet the continuous monitoring of the data transmitted to the unit can be used. Such a situation is depicted schematically in FIG. 7, in which a person 1 is present in the interior of a house 20. The person 1 whose state of health must be monitored continuously and/or periodically in conjunction with various acquired data, is for example on the top floor within the house 20. Again by means of sensors 2, 3′ and 6 various medical or physiologically relevant data are acquired. Via RF transmission, such as for example so-called Bluetooth transmission, the data are transmitted to the independent separate evaluation or processing unit 22′, which is disposed on the lower floor. In order to prevent the energy draw from the apparatus batteries, this evaluation and processing unit 22′ is connected to an electric wall socket 23. It is possible that therein simultaneously rechargeable storage media in the evaluation unit 22′ can be charged. In the event of a discrepancy of the data transmitted to it by the evaluation unit 22′ a signal or a further transmission of data can be generated, which are transmitted via a wire connection or wirelessly to a land line telephone apparatus 25. From this land line telephone apparatus 25 the appropriate signal or the data generated by the processing unit 22′ can be transmitted to a help center via a connection cable 27 or a land line connection 29.

It is understood that it is possible, and, if appropriate, also preferred that the separate evaluation or processing unit 22 or 22′ is directly integrally installed in a mobile telecommunication apparatus, such as for example the mobile telephone 5 according to FIG. 4. However, it is also possible that the evaluation unit 22 or 22′ can be connected via plugs, snaps or other types of connections with a mobile telecommunication apparatus 5, in order to form a unit in this manner.

It is understood, that the diagrams depicted in FIGS. 1 to 7, are only examples in order to explain the present invention in more detail. The elements selected in the diagrams as well as the described transmission technologies follow technologies and capabilities conventionally employed today. In particular mobile telephones with integrated GPS system have only recently come on the market and are only offered by a few manufacturers, such as already cited, for example the companies Benefon, Nokia, Ericsson and Motorola. However, it must also be assumed that such apparatus will be offered in the near future by other manufacturers also. It is also important that sufficient bandwidth for the data transmission from mobile telecommunication networks will be available. In this connection reference is made to GPRS and UMTS, which considerably increase the transmission rate.

Appendix: International Patent Application No. PCT/CH02/00247

1. Configuration for the acquisition and/or monitoring of medical data, in particular the state of the cardiovascular system, blood values or blood composition, electrocardiographic data, etc., characterized by
at least one measuring sensor (3, 3′) for the acquisition of the medical data, such as the state of the cardiovascular system, etc. of a person (1), comprising at least one light source, which can emit light at least at two frequencies, as well as at least one light receiver for determining the light transmitted through a tissue portion of the person,
an EKG measuring configuration (2) for the acquisition and monitoring of the cardiovascular state of a person,
if appropriate, an evaluation unit for the detection of potential irregularities of the data acquired by the measuring sensor or the EKG measuring system, and
a sending and receiving device (5) for voice and/or data, to address, if appropriate, at least a third party (9, 13, 19) and to transmit data to this party.

2. Configuration for the acquisition and/or monitoring of the state of health or of the medical data, such as for example the cardiovascular state, blood values or blood compositions, electrocardiographic data, etc., of a person, characterized by
at least one measuring sensor (3, 3′, 6) and/or at least one measuring configuration (2) for the acquisition of medical data or such data relevant to the state of health of the person (1),
at least one evaluation unit (22, 22′) comprising at least one logic control or digital electronic circuitry for signal processing and interpretation, inter alia for the evaluation and optionally for the detection, if appropriate, of irregularities of the data acquired by the measuring sensor and/or the measuring configuration, which evaluation unit is disposed spaced apart from the measuring sensor or the measuring configuration, and
a sending and receiving device (5, 25) for voice and/or data in order to address optionally a third party (9, 13, 19) and to transmit data to this party.

3. Configuration as claimed in claim 2, characterized in that
at least one measuring sensor (3, 3′) is provided for the acquisition of medical data, such as the state of the cardiovascular system of the person (1), comprising at least one light source, which can emit light at least at two frequencies, as well as at least one light receiver for determining the light transmitted through a tissue portion of the person, as well as
at least one EKG measuring configuration (2) for the acquisition and monitoring of the state of the cardiovascular system of a person or for the monitoring of the electrocardiographic functions of the person.

4. Configuration, in particular as claimed in one of claims 1 to 3, characterized in that further a locating system module is provided, by means of which the location of the person is transmitted to the third party.

5. Configuration, in particular as claimed in one of claims 1 to 4, characterized in that one or several measuring sensors or measuring configurations are provided for the acquisition of as many relevant medical data as possible, such as heart rate, respiratory rate, oxygen saturation of the blood, heart minute volume, blood sugar level, chest pressure (chest effort), body posture, snoring (snore sensor) and/or body temperature.

6. Configuration as claimed in one of claims 1 to 5, characterized in that the EKG measuring configuration for the acquisition and monitoring of the electrocardiographic functions, for the acquisition of a so-called three-leader preferably comprises at least four electrodes and twelve electrodes for the acquisition of a so-called twelve-leader.

7. Configuration as claimed in claim 6, characterized in that an evaluation system is provided for the evaluation of the electrocardiographic signals registered by the electrodes, in which evaluation system a personal EKG reference state is stored, with which the EKG signals registered by the electrodes are continuously compared and discrepancies, if appropriate, are transmitted to the sending and receiving device, with the evaluation system preferably being disposed in the evaluation or processing unit (22, 22′).

8. Configuration, in particular as claimed in one of claims 1 to 7, characterized in that as the sending and receiving unit (5) serves a telecommunication apparatus such as a mobile telephone (5), a land line dial-up station (25), an Internet dial-up station, etc.

9. Configuration, in particular as claimed in one of claims 1 to 8, characterized in that on the sending and receiving unit (5) communication and control electronic circuitry is disposed or integrated, which is connected to the dial element such that one or several preprogrammed telephone numbers and/or Internet addresses are dialed, and that from the sending unit, apart from measuring data, also position coordinates such as GPS (Global Positioning System) coordinates, are transmitted to the third party.

10. Configuration, in particular as claimed in one of claims 1 to 9, characterized in that for the transmission of the data from or to the measuring sensor (3, 3′, 6) and/or from or to the EKG measuring system (2) or optionally from the evaluation system to the evaluation unit (22, 22′) and/or to or from the sending and receiving device (5) as well as the transmission of the data from or to the evaluation unit (22, 22′) to or from the sending and receiving unit (5) takes place in the radio wave range, such as for example the so-called “Bluetooth” technology or wirelessly in another suitable transmission frequency and/or utilizing another protocol.

11. Configuration as claimed in one of claims 1 to 10, characterized in that at the third party a device (11, 12, 15) is provided, on which the acquired data measured by the measuring sensor or by the EKG measuring configuration (2) can be represented or visualized, as well as the location of the person to be monitored.

12. Configuration as claimed in one of claims 1 to 11, characterized in that the sending and receiving device as well as the device disposed at the third party are such that data and voice communication in both directions simultaneously is possible in order to make possible also voice communication during data transmissions between the person and the receiver.

13. Configuration, in particular as claimed in one of claims 1 to 12, characterized in that the measuring sensor
is a device (3) which can be placed on an ear, which comprises at least one part each placeable on one site of the ear lobe and/or of the outer ear, with
one part comprising a structural part for light emission and
the other part a light sensor or a light receiver, for determining the light transmitted through the lobe or the outer ear, and with
a sender being provided for the wireless transmission of the values determined by the sensor or evaluation data derived therefrom to the evaluation or processing unit (22, 22′) and/or, if appropriate, to the sending and receiving device.

14. Configuration as claimed in one of claims 1 to 13, characterized in that the EKG measuring configuration (2), respectively the electrodes, for the registration of the EKG are disposed in a chest belt (4) or are supported by it.

15. Configuration as claimed in claim 13, characterized in that in the chest belt (4) is additionally disposed the evaluation system, which is connected with the electrodes or the EKG measuring system.

16. Configuration as claimed in one of claims 1 to 15, characterized in that in the evaluation system a regulation function or logic is provided, in order to compare continuously the measured INSTANTANEOUS state with the targeted REFERENCE state and, in the presence of irregularities or discrepancies, continuously a dosed stimulant or supply of a relevant material/substance is delivered which affects the INSTANTANEOUS state.

17. Method for the acquisition and/or monitoring of the state of health or of medical data, such as for example of the state of the cardiovascular system and/or of the blood sugar level of a person by means of a configuration, in particular as claimed in one of claims 1 to 16, characterized in that
by means of at least one measuring sensor (3) on the person (1) the medical condition, in particular the state of the cardiovascular system is monitored,
optionally by means of an evaluation unit, comprising at least one logic control or digital electronic circuitry for signal processing and interpretation, irregularities of the acquired data are detected,
by means of at least one EKG measuring configuration (2) comprising at least four, preferably twelve, electrodes, an EKG is continuously registered,
optionally by means of an evaluation system irregularities or discrepancies of the EKG from a personal reference curve are detected or determined,
at least in the event of irregularities by means of a sending and receiving device for voice and/or data (5) optionally a third party is addressed and data are transmitted, as well as
by means of a locating or navigation system, such as GPS (Global Positioning System) the position of the person is transmitted to the third party.

18. Method for the acquisition and/or monitoring of the state of health or of medical data of a person by means of a configuration, in particular as claimed in one of claims 2 to 16, characterized in that by means of at least one measuring sensor (3, 3′, 2) and/or of a measuring device (2) on the person (1) the medical condition is monitored, the data acquired by the measuring sensor or the measuring device are transmitted to an evaluation unit (22, 22′) in which the acquired data are evaluated or by means of a software algorithm, stored for example in a logic circuit or network, irregularities of the acquired data are detected, at least in the event that irregularities or discrepancies of the acquired data from corresponding personal reference data are detected or determined, a signal or data stream is generated and transmitted to the sending and receiving device for voice and/or data (5), by which, if appropriate, a third party is addressed and the data are transmitted.

19. Method, in particular as claimed in claim 17 or 18, characterized in that the transmission of the data from the measuring sensor or from the EKG measuring configuration or the evaluation electronic circuit to the sending and receiving device is carried out by means of radio waves, such as for example so-called “Bluetooth” or with another frequency or another protocol.

20. Method, in particular as claimed in one of claims 17 to 19, characterized in that as the sending and receiving device (5) a GSM apparatus (Global System for Mobile Communication), a GPRS apparatus (General Packet Radio Service), a UMTS apparatus (Universal Mobile Telecommunication System), etc. is employed, which, based on a signal through the evaluation unit automatically addresses at least one third party and transmits data.

21. Method, in particular as claimed in one of claims 17 to 20, characterized in that the simultaneous data and voice communication between sending and receiving device (5) and the third party in both directions is possible in order for the third party to be able to establish contact with the person or, if appropriate, can read on the person data at the measuring sensor and/or from the EKG measuring configuration or the evaluation electronic circuit or can influence the measuring sensor or the evaluation system or other devices disposed at the patient.

22. Method as claimed in one of claims 17 to 21, characterized in that the EKG or the signals registered by the electrodes are transmitted in a chest belt worn by the person in analog or digital form via radio frequency to a processing module or the evaluation system, which processing module carries out the curve analysis in real time or quasi-real time and triggers an alarm if the processing module detects a discrepancy from a personal reference curve.

23. Method as claimed in one of claims 17 to 22, characterized in that the EKG measuring configuration checks continuously whether or not the registered EKG curve deviates in the P, Q, R, S and T characteristics from the personal normal course, and, in the event discrepancies are detected, an alarm is triggered.

24. Method as claimed in claim 23, characterized in that the evaluation unit analyzes the curve segments conventionally meaningful in cardiology, such as the QRS complex, the R-R interval, the S-T segment and the P-Q segment, where the following definitions apply:

| | |
|---|---|
| P: | depolarization atria |
| QRS: | depolarization ventricles |
| T: | repolarization atria and ventricles |
| P-Q: | conduction atria to ventricles |
| R-R: | indication of rate and type of rhythm. |

25. Method as claimed in one of claims 17 to 24, characterized in that a journal or a so-called event log for the monitored patients is kept, in which special events of all connected sensor or measuring configurations or communication configurations are recorded.

26. Method as claimed in one of claims 17 to 25, characterized in that the correct position of the measuring sensor or the sensors and/or the EKG measuring configuration or of the electrodes on the person or on the patient are continuously monitored and discrepancies from the correct position are detected and the patient, for example by means of the sending and receiving device, is informed of them and/or an alarm is triggered.

27. Use of the configuration as claimed in one of claims 1 to 16 for monitoring a person with cardiovascular disorders.

28. Use of the configuration as claimed in one of claims 1 to 16 for the acquisition of medical sports data, if appropriate by the person engaged in the sports activity himself.

29. Use of the configuration as claimed in one of claims 1 to 16 for monitoring the health of persons who have a risk constellation for cardiovascular diseases.

30. Use of the configuration as claimed in one of claims 1 to 16 for monitoring the blood sugar level in the case of diabetics optionally by the person suffering from diabetes himself.

31. Use of the configuration as claimed in one of claims 1 to 16 for the monitoring of patients whose pulmonary function is impaired.

32. Use as claimed in claim 31 for the regulation of the oxygen supply to a person suffering from oxygen insufficiency in the blood, wherein by means of a regulation logic in the evaluation unit the oxygen supply to the patient is regulated or optimally dosed.

33. Use of the configuration as claimed in one of claims 1 to 16 for the monitoring of infants or babies.

34. Use of the configuration as claimed in one of claims 1 to 16 for the medical monitoring of patients in a dentist's office, in particular during dental surgery of persons with medical disorders.
